SEQUENCE LISTING (24 sequences)

SEQ ID NO: 1 - protein, 11 aa, Artificial Sequence
  oligopeptide C19-29 region of glucagon of human, mouse or rat
AQDFVQWLMNT

SEQ ID NO: 2 - DNA, 1471 bp, Artificial Sequence
  DNA insert encoding rat IFN-r receptor, rat IgG Fc region and glucagon C19-29 region
gaattcatttaaatgattctgctggtggtcctgatgctgtctgcggagatcgggagtggagctttgatgagcaccgaggatcctaagccgccctcggtgcctgcgccaacaaatgttctaattacgtcctatgacttgaaccctgtcgtacattggaagcaccagaacgtgtcgcaggctgccgtcttcactgtacaggtaaagatgtatccagaatactggactgatgcctgcaccaacattgcccatcattattgtaatatctacaaacacatttcctatcctgactcatctgcctgggccagagttaaggccaaggttggacaaagagaatctgcctatgcgcagtcagaagagtttattatgtgccgaaaggggaaggttggaccgcctggcctggacatcggaaggaaggaagatcagctgattgtccacatatttcaccctaaggtcaatgtgagtcaggaaaccatgtttggtgacggaaatacctgttacacattcgactacactgtgtttgtgaaacattacaggagtggggagatcctacatacagaacatagcgtcctaaaagaagattgtagcgaaactctgtgtgagttaaacatctcagtgtccacgctgaattccaattactgtgtttcagtagttggaaagtcgtctttctggcaagttaatacagaaacatcaaaagacgcctgtatcccctttctccatgatgacagagaagaagcggccgccgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgacccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaagcccaagattttgtgcagtggttgatgaatacctgagaattct

SEQ ID NO: 3 - DNA, 4790 bp, Artificial Sequence
  DNA sequence of artificial expression vector pCAGGS
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcctcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 4 - DNA, 1233 bp, Artificial Sequence
  DNA insert encoding rat CTLA4, rat IgG Fc region and glucagon C19-29 region
gaattcatttaaatggcttgtcttggactccagaggtacaaaactcacctgcagctgccttctaggacttggccttttggagtcctgctttctcttctcttcatcccaatcttctctgaagccatacaagtgacccaaccttcagtggtgttggccagcagccacggtgtcgccagctttccatgtgaatatgcatcttcacacaacactgatgaggtccgggtgacggtgctgcggcagacaaatgaccaagtgacagaggtctgtgccacgacattcacagtgaagaacacgttgggcttcctagatgaccccttctgcagtggtacctttaatgaaagcagagtgaacctcaccatccaaggactgagggctgctgacaccggactgtacttctgcaaggtggaactcatgtacccaccgccatactttgtgggcatgggcaacgggacccagatttatgtcatcgatccagaaccatgcccagattcagacgcggccgccgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgacccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaagcccaagattttgtgcagtggttgatgaatacctgagaattc

SEQ ID NO: 5 - DNA, 1143 bp, Artificial Sequence
  DNA insert encoding rat IL13, rat IgG Fc region and glucagon C19-29 region
gaattcatttaaatggcactctgggtgactgcagtcctggctctcgcttgccttggtggtcttgccaccccagggccagtgcggagatccacatctccccctgtggccctcagggagcttatcgaggagctgagcaacatcacacaagaccagaagacttccctgtgcaacagcagcatggtatggagcgtggacctgacagctggcgggttctgtgcagccctggaatccctgaccaacatctccagttgcaatgccatccacaggacccagaggatattgaatggcctctgtaaccaaaaggcctcggatgtggcttccagccccccagataccaaaatcgaagtagcccagtttatatcaaaactgctcaattactccaagcaacttttccgctatggccacgcggccgccgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgacccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaagcccaagattttgtgcagtggttgatgaatacctgagaattc

SEQ ID NO: 6 - DNA, 825 bp, Artificial Sequence
  DNA insert encoding rat signal peptide, rat IgG Fc region and glucagon C19-29 region
gaattcatttaaatgaagtcctgcggcctgttccctctcatggtgctccttgctctgggtgtactggcaccctggagtgtggaaggagcggccgccgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgacccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaagcccaagattttgtgcagtggttgatgaatacctgagaattc

SEQ ID NO: 7 - DNA, 1284 bp, Artificial Sequence
  DNA insert encoding rat IL1 receptor antagonist, rat IgG Fc region and glucagons C19-29 region
gaattcatttaaatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaagcggccgccgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgacccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaagcccaagattttgtgcagtggttgatgaatacctgagaattc

SEQ ID NO: 8 - DNA, 369 bp, Artificial Sequence
  DNA insert encoding human IL8 and glucagon C19-29 region
gaattcatttaaatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcagcggccgccccgggtaaagcccaagattttgtgcagtggttgatgaatacctgagaattc

SEQ ID NO: 9 - DNA, 45 bp, Artificial Sequence
  Oligonucleotide primer used for constructing a vector pCAGGS-IgG-glu19-29
gagaattcatttaaatgagagcggccgccgtgcccagaaactgtg

SEQ ID NO: 10 - DNA, 49 bp, Artificial Sequence
  Oligonucleotide primer used for constructing a vector pCAGGS-IgG-glu19-29
tcaaccactgcacaaaatcttgggctttacccggagagtgggagagact

SEQ ID NO: 11 - DNA, 45 bp, Artificial Sequence; identical to SEQ ID NO: 9
  Oligonucleotide primer used for constructing a vector pCAGGS-IgG-glu19-29

SEQ ID NO: 12 - DNA, 50 bp, Artificial Sequence
  Oligonucleotide primer used for constructing a vector pCAGGS-IgG-glu19-29
gagagagagaattctcaggtattcatcaaccactgcacaaaatcttgggc

SEQ ID NO: 13 - DNA, 38 bp, Artificial Sequence
  Oligonucleotide primer used for constructing a vector pCAGGS-IFN-rR-IgG-glu19-29
gagaattcatttaaatgattctgctggtggtcctgatg

SEQ ID NO: 14 - DNA, 40 bp, Artificial Sequence
  Oligonucleotide primer used for constructing a vector pCAGGS-IFN-rR-IgG-glu19-29
gcagcatcgcggccgcttcttctctgtcatcatggagaaa

SEQ ID NO: 15 - DNA, 38 bp, Artificial Sequence
  Oligonucleotide primer used for constructing a vector pCAGGS-CTLA4-IgG-glu19-29
gagaattcatttaaatggcttgtcttggactccagagg

SEQ ID NO: 16 - DNA, 40 bp, Artificial Sequence
  Oligonucleotide primer used for constructing a vector pCAGGS-CTLA4-IgG-glu19-29
gcagcatcgcggccgcgtctgaatctgggcatggttctgg

SEQ ID NO: 17 - DNA, 38 bp, Artificial Sequence
  Oligonucleotide primer used for constructing a vector pCAGGS-IL13-IgG-glu19-29
gagaattcatttaaatggcactctgggtgactgcagtc

SEQ ID NO: 18 - DNA, 40 bp, Artificial Sequence
  Oligonucleotide primer used for constructing a vector pCAGGS-IL13-IgG-glu19-29
gcagcatcgcggccgcgtggccatagcggaaaagttgctt

SEQ ID NO: 19 - DNA, 38 bp, Artificial Sequence
  Oligonucleotide primer used for constructing a vector pCAGGS-IL1RA-IgG-glu19-29
gagaattcatttaaatggaaatctgctggggaccctac

SEQ ID NO: 20 - DNA, 40 bp, Artificial Sequence
  Oligonucleotide primer used for constructing a vector pCAGGS-IL1RA-IgG-glu19-29
gcagcatcgcggccgcttggtcttcctggaagtagaactt

SEQ ID NO: 21 - DNA, 62 bp, Artificial Sequence
  Oligonucleotide primer used for constr ucting a vector pCAGGS-glu19-29
gagaattcatttaaatgagagcggccgccccgggtaaagcccaagattttgtgcagtggttg

SEQ ID NO: 22 - DNA, 50 bp, Artificial Sequence; identical to SEQ ID NO: 12
  Oligonucleotide primer used for constructing a vector pCAGGS-glu19-29

SEQ ID NO: 23 - DNA, 38 bp, Artificial Sequence
  Oligonucleotide primer used for constructing a vector pCAGGS-IL8-glu19-29
gagaattcatttaaatgacttccaagctggccgtggct

SEQ ID NO: 24 - DNA, 40 bp, Artificial Sequence
  Oligonucleotide primer used for constructing a vector pCAGGS-IL8-glu19-29
gcagcatcgcggccgctgaattctcagccctcttcaaaaa

(19) United States
(12) Patent Application Publication
(10) Pub. No.: US 2006/0223767 A1
(43) Pub. Date: Oct. 5, 2006

(54) Vector for gene therapy and method of quantifying target protein in mammal or cultured cells with the administration of the vector for gene therapy

(76) Inventor: Haruo Hanawa, Niigata-shi (JP)

Correspondence Address: BIRCH STEWART KOLASCH & BIRCH, PO BOX 747, FALLS CHURCH, VA, 22040-0747, US

(21) Appl. No.: 10/541,626

(22) PCT Filed: Dec. 26, 2003
(86) PCT No.: PCT/JP03/16956

§ 371 Date: Jul. 7, 2005

(30) Foreign Application Priority Data
Jan. 10, 2003 (JP) 20033967

(51) Int. Cl. A61K 48/00; G01N 33/53; C07K 14/52; C07K 14/54; C07K 14/56; C07K 14/715

(52) U.S. Cl. 514/44; 435/7.1; 530/395; 530/397; 530/351

(57) ABSTRACT

Disclosed is a vector for gene therapy, by which the blood level of a desired protein can be monitored at high sensitivity when a gene therapy is conducted, wherein the label peptide does not have a physiological activity and has no immunogenicity in many mammals. The vector for gene therapy comprises a nucleic acid coding for a fusion protein of glucagon C-terminal side 19-29 amino acid peptide region and a desired protein region which should be produced in the body, which nucleic acid is incorporated into an expression vector for mammalian cells.

### TECHNICAL FIELD

The present invention relates to a vector for gene therapy for the production of a desired protein in the body, which vector enables to simply quantify the desired protein produced in the body or in cultured cells.

### BACKGROUND ART

When a gene therapy by which a desired protein is made to be produced in the body of a patient, the blood level of the desired protein cannot be measured unless a method for measuring the protein, such as ELISA, has been established. To counter this problem, an assay method in which a label protein is used and the level of the protein is measured has been practically used and sold. However, the measurement sensitivity of the method is low, and so there is no established method for measuring blood level in gene therapy. In Treatment of Murine Lupus with cDNA encoding IFN-γ R/Fc, The Journal of Clinical Investigation, July 2000, volume 106, Number 2 p207-215, the desired protein is not measured but a protein influenced by the desired protein is quantified so as to indirectly prove the expression of the desired protein. This is thought to show the difficulty in the measurement of the blood level.

### DISCLOSURE OF THE INVENTION

An object of the present invention is to provide a vector for gene therapy, by which the blood level of a desired protein can be monitored at high sensitivity when a gene therapy is conducted, and by which undesired physiological action or antigen-antibody reaction due to the label scarcely occurs.

The present inventor intensively studied to discover that by carrying out the gene therapy with a vector in which a fusion protein of the desired protein which should be produced in the body by the gene therapy and a glucagon C-terminal side 19-29 amino acid peptide, the blood level of the desired protein may be measured with high sensitivity using the glucagon peptide as a label, and that undesired physiological action or induction of immunological reaction due to the label peptide scarcely occurs, thereby completing the present invention.

That is, the present invention provides a vector for gene therapy comprising an expression vector for mammalian cells and a nucleic acid coding for a fusion protein of glucagon C-terminal side 19-29 amino acid peptide region and a desired protein region which should be produced in the body, which vector can produce the fusion protein in the mammalian cells. The present invention also provides a method for gene therapy comprising administering an effective amount of the vector for gene therapy according to the present invention to a mammal or cultured mammalian cells, in which expression of the desired protein in the body or in the cultured mammalian cells is desired. The present invention further provides a use of the vector for gene therapy according to the present invention for the production of a drug for gene therapy. The present invention still further provides a method for quantifying a desired protein produced in the body or in cultured cells by expression of the vector for gene therapy, comprising quantifying, by immunoassay, the glucagon C-terminal side 19-29 amino acid peptide region in a test sample collected from a mammal or cultured mammalian cells to which the vector for gene therapy according to the present invention was administered. The present invention still further provides a label for labeling a desired protein produced by expression of an externally administered expression vector in the body of a mammal or in cultured mammalian cells, consisting essentially of glucagon C-terminal side 19-29 amino acid peptide. The present invention still further provides a method for labeling a protein produced in the body or in cultured cells, comprising labeling a desired protein produced in the body or in cultured cells with glucagon C-terminal side 19-29 amino acid peptide by expressing the desired protein produced by expression of an externally administered expression vector in the body of a mammal or in cultured mammalian cells, as a fusion protein with the glucagon C-terminal side 19-29 amino acid peptide as a label. The present invention still further provides a use of glucagon C-terminal side 19-29 amino acid peptide as a label for a desired protein produced by expression of an externally administered expression vector in the body of a mammal or in cultured mammalian cells.

By the present invention, a vector for gene therapy by which the blood level of the desired protein can be measured with high sensitivity, wherein the label peptide does not have a physiological action, was first provided. Since the glucagon C-terminal side 19-29 itself does not have a physiological action and is well conserved in various mammals, it does not substantially induce an immunological reaction while it can be quantified by immunoassay with high sensitivity using a commercially available immunoassay kit.

### BRIEF DESCRIPTION OF THE DRAWINGS

FIG. 1 shows the nucleotide sequence of the nucleic acid fragment inserted into the vector for gene therapy prepared in Example 1, together with the amino acid sequence encoded thereby.

FIG. 2 shows the continuation of FIG. 1.

FIG. 3 shows the continuation of FIG. 2.

FIG. 4 shows a gene map of pCAGGS which is an expression vector for mammals used in Examples 1 to 5.

FIG. 5 shows the relationship between the days after administration of the vector for gene therapy and the blood level of the desired protein measured by measuring the glucagon-originated label peptide, in Example 1.

FIG. 6 shows the change with time of the blood level of the desired protein when gene therapy was carried out using the vector of the present invention, and shows the change with time of the blood glucose level when gene therapy was carried out using the vector of the present invention or using a vector in which a nucleic acid coding for the desired protein not fused with the glucagon-originated label peptide was inserted.

FIG. 7 shows the nucleotide sequence of the nucleic acid fragment inserted into the vector for gene therapy prepared in Example 2, together with the amino acid sequence encoded thereby.

FIG. 8 shows the continuation of FIG. 7.

FIG. 9 shows the continuation of FIG. 8.

FIG. 10 shows the relationship between the days after administration of the vector for gene therapy and the blood level of the desired protein measured by measuring the glucagon-originated label peptide, in Example 2.

FIG. 11 shows the days for which the transplanted hearts were taken by the rats in Example 2 and Comparative Example 2.

FIG. 12 shows the nucleotide sequence of the nucleic acid fragment inserted into the vector for gene therapy prepared in Example 3, together with the amino acid sequence encoded thereby.

FIG. 13 shows the continuation of FIG. 12.

FIG. 14 shows the continuation of FIG. 13.

FIG. 15 shows the nucleotide sequence of the nucleic acid fragment inserted into the vector for gene therapy prepared in Comparative Example 3, together with the amino acid sequence encoded thereby.

FIG. 16 shows the continuation of FIG. 15.

FIG. 17 shows the relationship between the days after administration of the vector for gene therapy and the blood level of the desired protein measured by measuring the glucagon-originated label peptide, in Example 3.

FIG. 18 shows the percentage of the lesion area of myocarditis in rats in Example 3 and Comparative Example 3.

FIG. 19 shows the nucleotide sequence of the nucleic acid fragment inserted into the vector for gene therapy prepared in Example 4, together with the amino acid sequence encoded thereby.

FIG. 20 shows the continuation of FIG. 19.

FIG. 21 shows the continuation of FIG. 20.

FIG. 22 shows the relationship between the days after administration of the vector for gene therapy and the blood level of the desired protein measured by measuring the glucagon-originated label peptide, in Example 4.

FIG. 23 shows the percentage of the lesion area of rat myocarditis in Example 4 and Comparative Example 4.

FIG. 24 shows the nucleotide sequence of the nucleic acid fragment inserted into the vector for gene therapy prepared in Example 5, together with the amino acid sequence encoded thereby.

FIG. 25 shows the relationship between the blood level of the desired protein measured by measuring the glucagon-originated label peptide and the blood level of the desired protein measured by measuring human interleukin 8, in Example 5.

### BEST MODE FOR CARRYING OUT THE INVENTION

The “glucagon C-terminal side 19-29 amino acid peptide” which is expressed by the vector according to the present invention in the form of a fusion protein, means the peptide consisting essentially of totally 11 amino acids located from the 19th to 29th amino acid counted from the C-terminal of glucagon. That is, the peptide has the amino acid sequence shown in SEQ ID NO: 1 in SEQUENCE LISTING. Since the “glucagon C-terminal side 19-29 amino acid peptide” is used as a label of the desired protein, the peptide may be hereinafter referred to as “glucagon-originated label peptide” for convenience.

In the vector according to the present invention, a nucleic acid coding for a fusion protein of the glucagon-originated label peptide region and a desired protein region is incorporated.

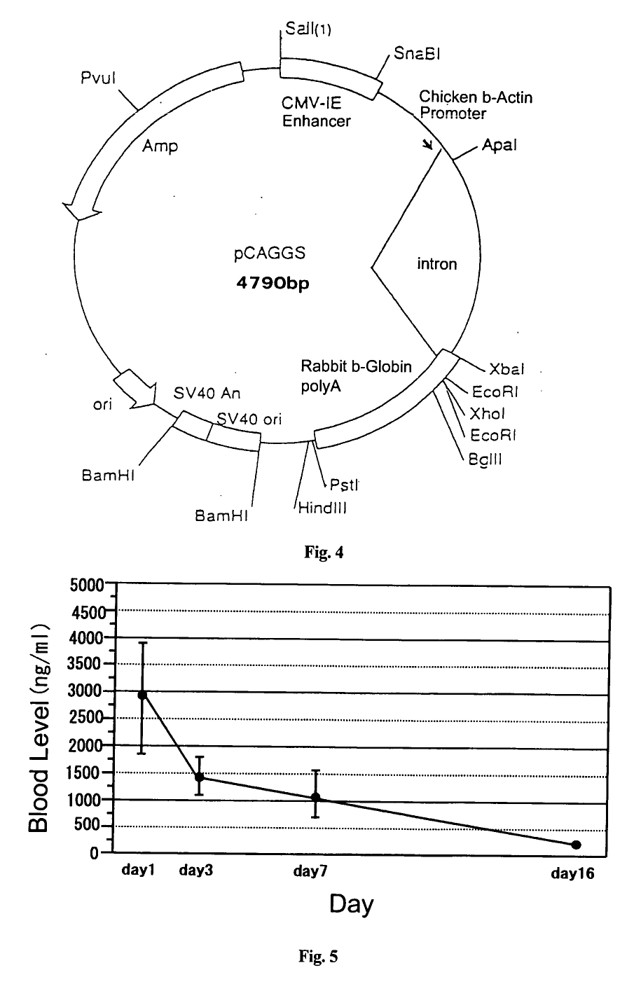

The expression vector for mammalian cells used in the present invention is well-known in the field of gene therapy, and is not restricted as long as it is an expression vector for mammalian cells. The characteristic feature of the present invention resides in that the glucagon-originated label peptide region as a label of the desired protein is expressed in the form of a fusion protein with the desired protein, so that the expression vector for mammalian cells is not restricted at all, and any of the known expression vectors for mammalian cells used in the field of gene therapy may be employed. Although the vector may be a plasmid vector or a virus vector, a plasmid vector is preferred from the view point of safety. Various expression vectors for mammalian cells are well-known and are commercially available, and the well-known or commercially available vectors may preferably be employed. Examples of the well-known or commercially available vectors include pCAGGS (Efficient selection for high expression transfactans with a novel eukaryotic vector, Gene Dec. 15, 1991, 108(2)p193-P199., whose gene map is shown in FIG. 4, and whose nucleotide sequence is shown in SEQ ID NO: 3 in SEQUENCE LISTING), pCI vectors, pSI vectors and pTARGET vectors from PROMEGA, and pcDNA5/TO and the like from INVITROGEN, but the vectors are not restricted thereto.

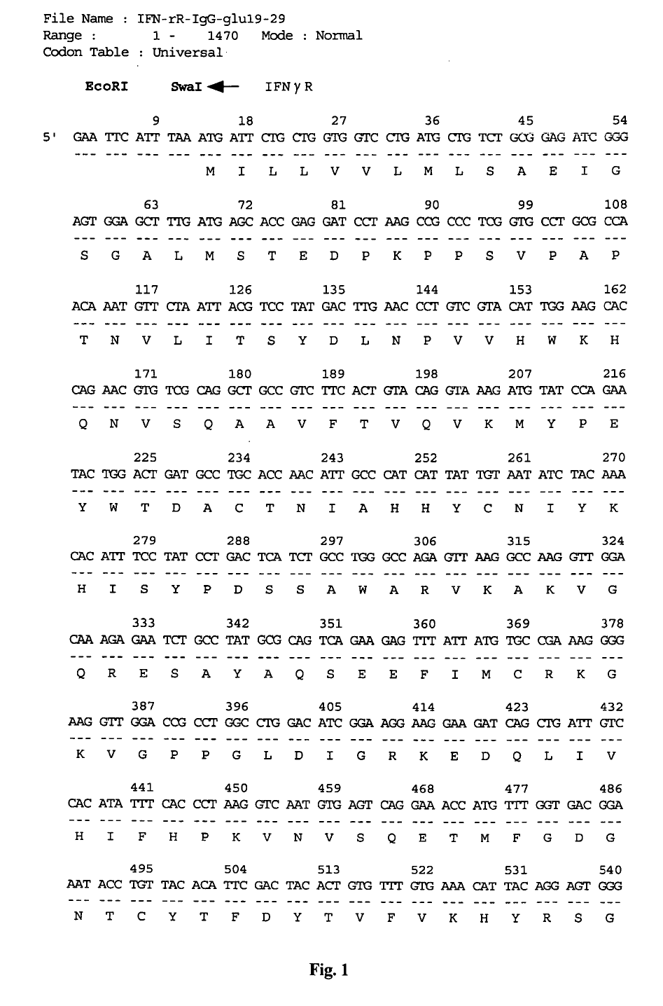

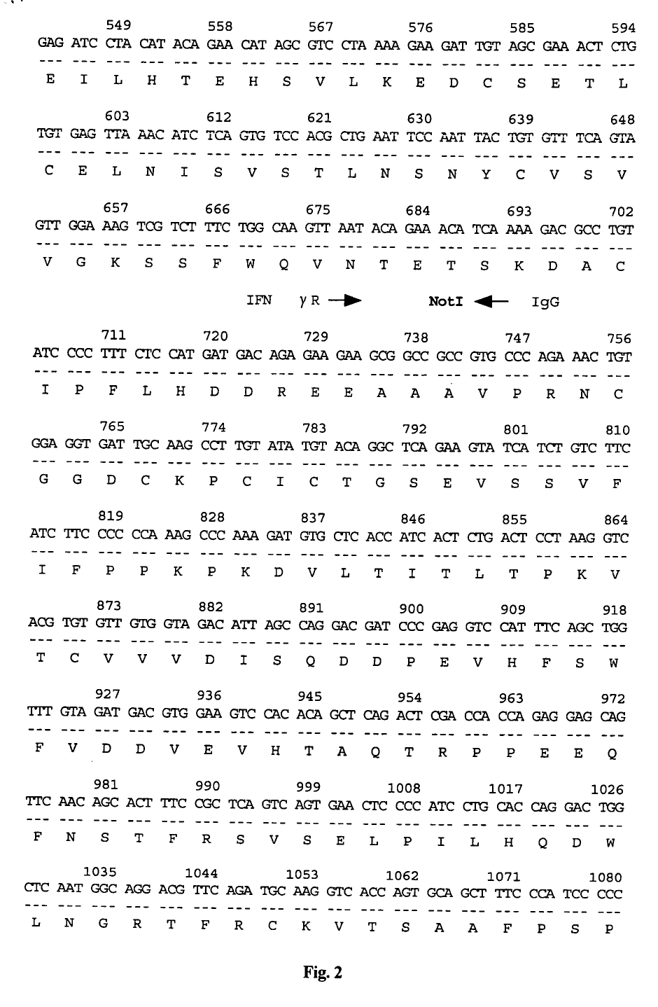

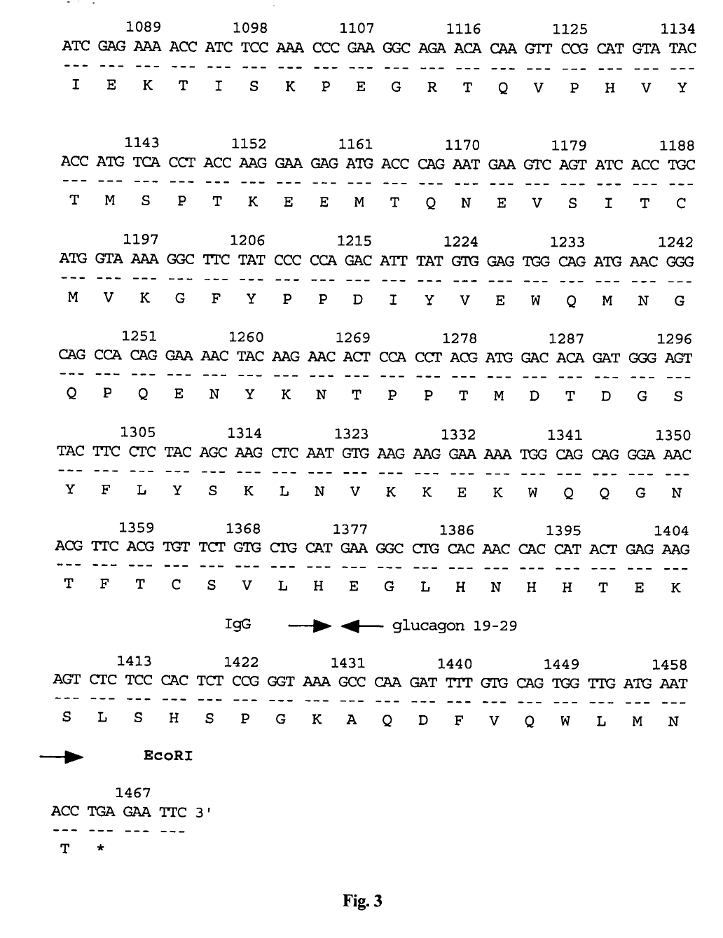

The desired protein to be produced in the body by the gene therapy is not restricted at all, and examples thereof include various cytokines such as interferons, interleukins and CTLA4; hormones such as growth factor and insulin; and cell adhesion factors; as well as receptors thereof. An arbitrary antigenic protein may also be produced in the body by a gene vaccine. The desired protein itself may also be a fusion protein. For example, a terminal of a desired cytokine which is desired to be bound to a Fc receptor may be fused with an immunoglobulin, preferably IgG, especially the constant region (Fc) of IgG1 so as to promote the binding ability with the Fc receptor, and the resulting fusion protein may be employed as the desired protein (see Examples 1 to 4 below). The nucleotide sequence coding for the Fc region of IgG is well-known. For example, the nucleotide sequence of the nucleic acid coding for the Fc region of human IgG is described in, for example, GenBank Accession No. BC020823 and so on, and the nucleotide sequence of the nucleic acid coding for the Fc region of rat IgG is shown in FIGS. 1 to 3 of the present application, and so on.

The vector according to the present invention may be obtained by inserting the nucleic acid coding for the fusion protein of the desired protein and the above-described label peptide into a cloning site of the expression vector for mammalian cells. It is preferred to fuse the label peptide to a terminal, especially to the C-terminal of the desired protein.

Gene therapy may be carried out by administering the vector for gene therapy according to the present invention to a mammal. As the administration route, a parenteral route such as intravenous injection or intramuscular injection is preferred. The dose of the vector may be appropriately selected depending on the properties of the desired protein, the type and the severity of the disease to be treated, and the dose of the vector per 1 kg of body weight may be usually about 1 mg to 10 mg, preferably about 2 mg to 4 mg. As for the formulation, the vector for gene therapy may be dissolved in Ringer solution and the obtained solution may be used as an injection solution. Additives for injection solution, well-known in the field of drug formulation may also be added. Alternatively, the vector for gene therapy according to the present invention may be administered to mammalian cells cultured in vitro. More particularly, there is a gene therapy in which the cells such as lymphocytes and bone marrow cells from a patient are collected from the body and cultured, a gene vector is administered to the cultured cells, and the cells acquired the ability to produce the desired protein are returned again to the patient. The vector for gene therapy according to the present invention may also be administered to such cultured mammalian cells. Alternatively, the vector for gene therapy may be administered to cultured mammalian cells in experiments for examining the therapeutic effect, in vitro, of the vector for gene therapy or the like.

In the gene therapy, the fusion protein of the above-described desired protein and the glucagon-originated label peptide is produced by the vector introduced into the body. Alternatively, in cases where the vector for gene therapy is administered to mammalian cells cultured in vitro, the fusion protein of the above-described desired protein and the glucagon-originated label peptide is produced in the cultured cells. Since the desired protein is fused with the label peptide, the level of the desired protein can be measured by measuring the level of the glucagon-originated label peptide. Since kits (including an antibody obtained by using the glucagon-originated label peptide as an antigen) for quantifying, by immunoassay, the glucagon-originated label peptide used in the present invention are commercially available (Pancreas Glucagon RIA kit commercially available from DAIICHI RADIOISOTOPE LABORATORIES, LTD and the like), the glucagon-originated label peptide may easily be measured using such a commercially available immunoassay kit.

The test sample for which the glucagon-originated label peptide is quantified may be various body fluids or tissues or dilutions thereof, collected from the individual to which the vector for gene therapy according to the present invention was administered, and preferably blood samples such as whole blood, serum or plasma as well as dilutions thereof. Alternatively, in cases where the vector for gene therapy is administered to the mammalian cells cultured in vitro, the test sample may be the homogenate of the cultured cell, culture supernatant or the like.

### EXAMPLE 1, COMPARATIVE EXAMPLE 1

PCR was carried out using as a template the cDNA of cultured rat spleen cells stimulated by lection, and using as primers 5′-gagaattcatttaaatgagagcggccgccgtgcccagaaactgtg-3′ and 5′-tcaaccactgcacaaaatcttgggctttacccggagagtgggagagact-3′. Thereafter, PCR was performed using as the template the 300-fold diluted PCR product obtained above, and using as primers 5′-gagaattcatttaaatgagagcggccgccgtgcccagaaactgtg-3′ and 5′-gagagagagaattctcaggtattcatcaaccactgcacaaaatcttgggc-3′. The amplified product was inserted into a cloning site of the above-described expression vector pCAGGS for mammalian cells. By this, pCAGGS-IgG-glu19-29 (pCAGGS in which a nucleic acid fragment having a region coding for the Fc region of immunoglobulin G1 (IgG1) and a region coding for the glucagon-originated label peptide, ligated to the downstream of the Fc-coding region, was inserted at the EcoRI site of pCAGGS), having restriction sites of SwaI and NotI, was obtained.

Thereafter, PCR was performed using as a template the cDNA of heart of rat suffering from myocarditis, and using as primers 5′-gagaattcatttaaatgattctgctggtggtcctgatg-3′ and 5′-gcagcatcgcggccgcttcttctctgtcatcatggagaaa-3′. The obtained PCR product was inserted into the pCAGGS-IgG-glu 19-29 prepared above using SwaI and NotI.

By this, a vector according to the present invention, in which the DNA fragment having a nucleotide sequence shown in SEQ ID NO:2 in SEQUENCE LISTING (shown together with the restriction sites) was inserted into the EcoRI site of the above-described expression vector pCAGGS for mammalian cells was prepared (Example 1). SEQ ID NO:2, together with other information, is shown in FIGS. 1 to 3. As shown in FIGS. 1 to 3, the inserted nucleic acid fragment (IFNγR-IgG-glucagon¹⁹⁻²⁹) had EcoRI sites at both ends, in which a region coding for the glucagon-originated label peptide was ligated to the downstream of the region coding for the fusion protein of interferon y receptor (IFNγR) protein and Fc region of immunoglobulin G1 (IgG1).

A plasmid vector not containing the glucagon-originated label peptide (Comparative Example 1) and the plasmid vector (Example 1) according to the present invention prepared as described above, which contained the glucagon-originated label peptide, were intravenously administered by jet injection to 7 rats, respectively, from the tail vein, thereby performing gene therapy. The injection solution was one obtained by dissolving 800 μg per rat of the plasmid dissolved in 20 ml of Ringer solution. After the injection, blood was sampled with time, the sampled 1 to 10 μl of plasma was 100-fold to 1000-fold diluted, and the level of the glucagon-originated label peptide, and in turn, the level of the desired protein (in this Example, the IFNγR/IgG1Fc fusion protein) was measured using a commercially available RIA kit (Pancreas Glucagon RIA kit commercially available from DAIICHI RADIOISOTOPE LABORATORIES, LTD and the like) in accordance with the instructions attached to the kit. That is, more concretely, RIA was performed as follows: To 400 μl of buffer for assay, 200 μl of standard glucagon solution or diluted test plasma was added, and 100 μl of glucagon-¹²⁵I solution and 100 μl of glucagon anti-serum solution were added, followed by leaving the resultant to stand at 4° C. for 48 hours. Thereafter, 100 μl of a second antibody and 400 μl of a precipitation stabilizer for glucagon RIA were added, and the resulting mixture was left to stand at 4° C. for 30 minutes. The resultant was then centrifuged (2000×g, 30 minutes, 4° C.), and the count rate was measured to determine the level after removing the supernatant.

FIG. 5 shows the results of the measurement of the blood level. In Example 1, on Day 1 after intravenous injection, the level was 2870±1062 ng/ml (mean±standard deviation), on Day 3, the level was 1440±334 ng/ml, on Day 7, the level was 1120±433 ng/ml, and on Day 16, the level was 281±162 ng/ml, and the level was able to be measured in all cases. On the other hand, in the gene therapy with the plasmid vector not containing the glucagon peptide (Comparative Example 1), the level was less than the detection limit in all cases by the same assay.

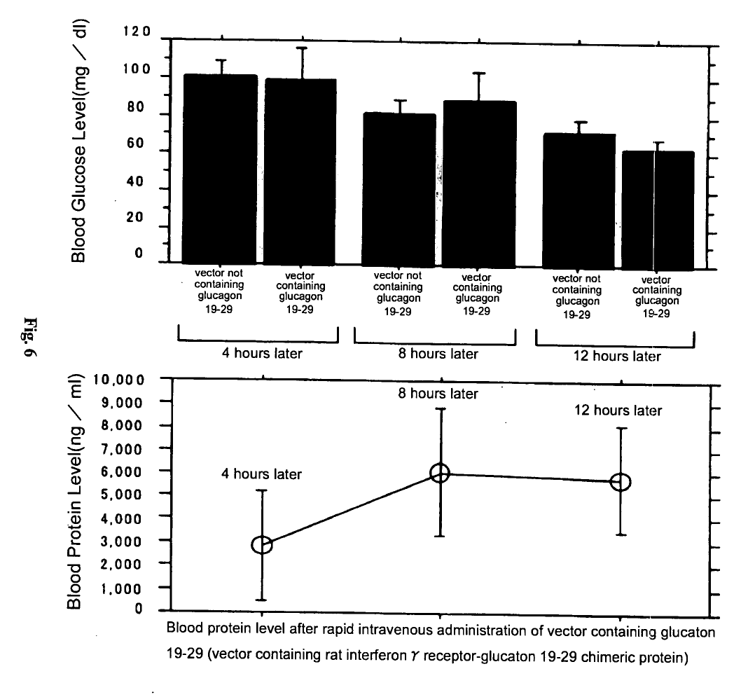

FIG. 6 shows blood glucose level and the blood protein level measured by the RIA as described above, at 4, 8 and 12 hours after the intravenous injection of the plasmid. In Example 1, the blood level was 2815±2318 ng/ml at 4 hours later, 6061±2789 ng/ml at 8 hours later, and 5752±2270 ng/ml at 12 hours later. The blood glucose levels at 8 to 12 hours later in which the blood protein level was the maximum, were 89.3±15.1 mg/dl (Example 1) vs. 81.8±7.5 mg/dl (Comparative Example 1), and 63.5±5.7 mg/dl (Example 1) vs. 71.4±6.9 mg/dl (Comparative Example 1), so that there were no differences.

From the results described above, it was shown that the blood level of the desired protein can be well measured until several tens days after the administration of the vector, using a very small quantity of plasma sample.

### EXAMPLE 2, COMPARATIVE EXAMPLE 2

PCR was carried out using as a template the cDNA of cultured rat spleen cells stimulated by lectin, and using as primers 5′-gagaattcatttaaatggcttgtcttggactccagagg-3′ and 5′-gcagcatcgcggccgcgtctgaatctgggcatggttctgg-3′. The obtained PCR product was incorporated into pCAGGS-IgG-glu19-29 prepared by the method described in Example 1, using SwaI and NotI.

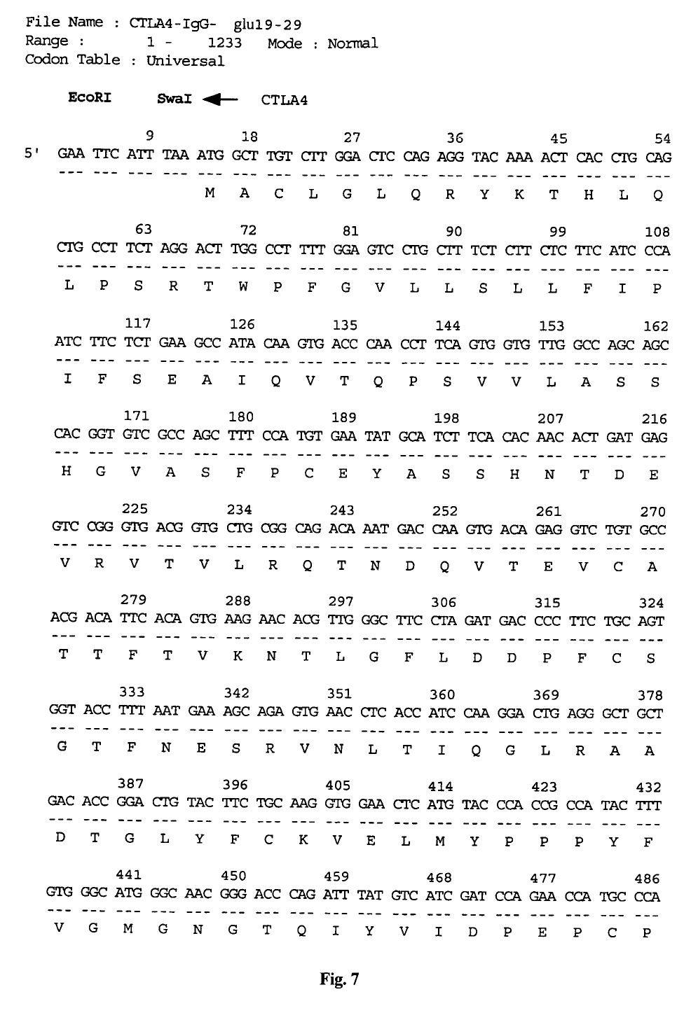

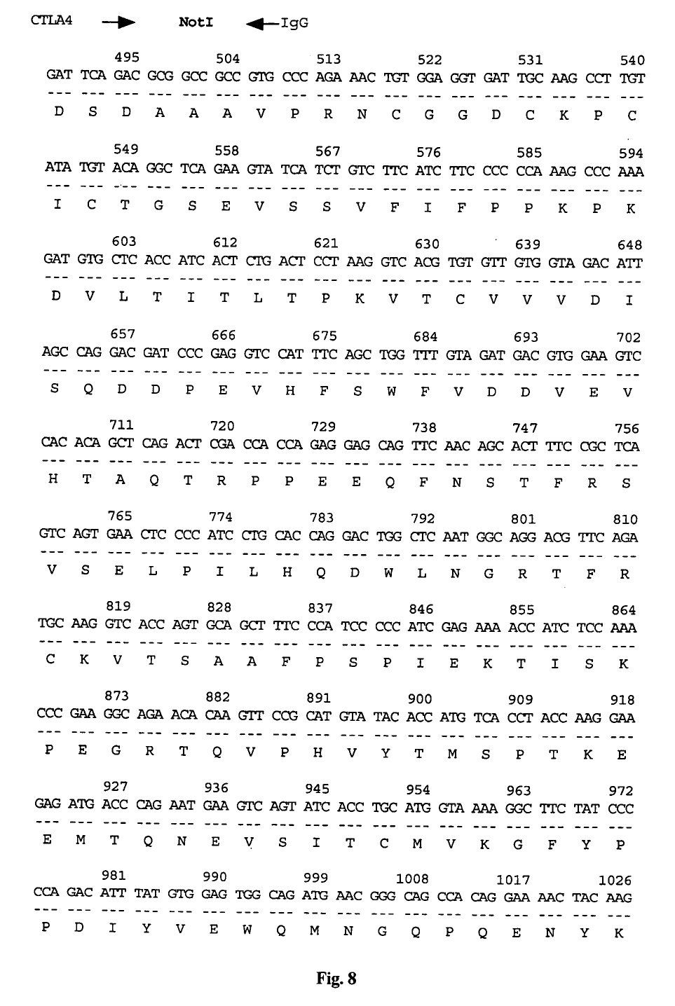

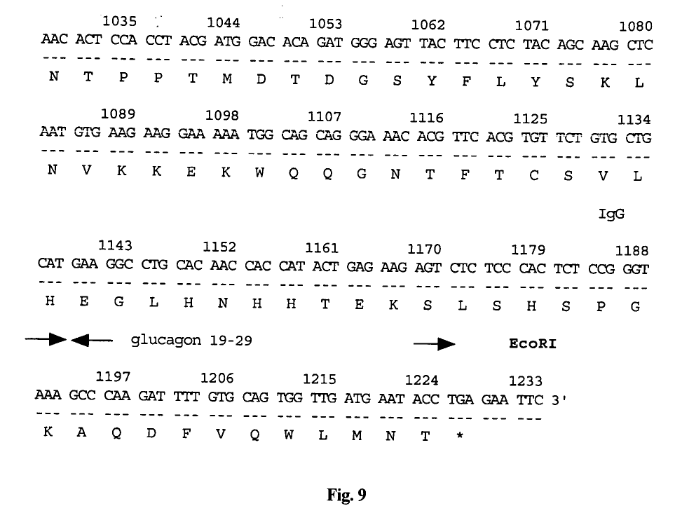

By this, a recombinant vector containing rat CTLA4-IgG-glucagon¹⁹⁻²⁹ (a nucleic acid fragment in which the rat IgG Fc-coding region was ligated to the downstream of rat CTLA4-coding region, and the glucagon-originated label peptide-coding region was ligated to the downstream of the rat IgG Fc-coding region) having the nucleotide sequence shown in FIGS. 7 to 9 and in SEQ ID NO:4 (the restriction sites are also shown), inserted into the EcoRI site of the above-described expression vector pCAGGS for mammalian cells, which recombinant vector expresses CTLA4-IgG-glucagon¹⁹⁻²⁹ in rat cells was prepared (Example 2). For comparison, a recombinant vector in which IgG-glucagon¹⁹⁻²⁹-coding region alone not containing the CTLA4-coding region was inserted was also prepared (Comparative Example 2).

To each rat after heart transplantation, the recombinant vector was administered in the same manner as in Example 1, and the blood level was measured. The days during which the heart was taken were also checked.

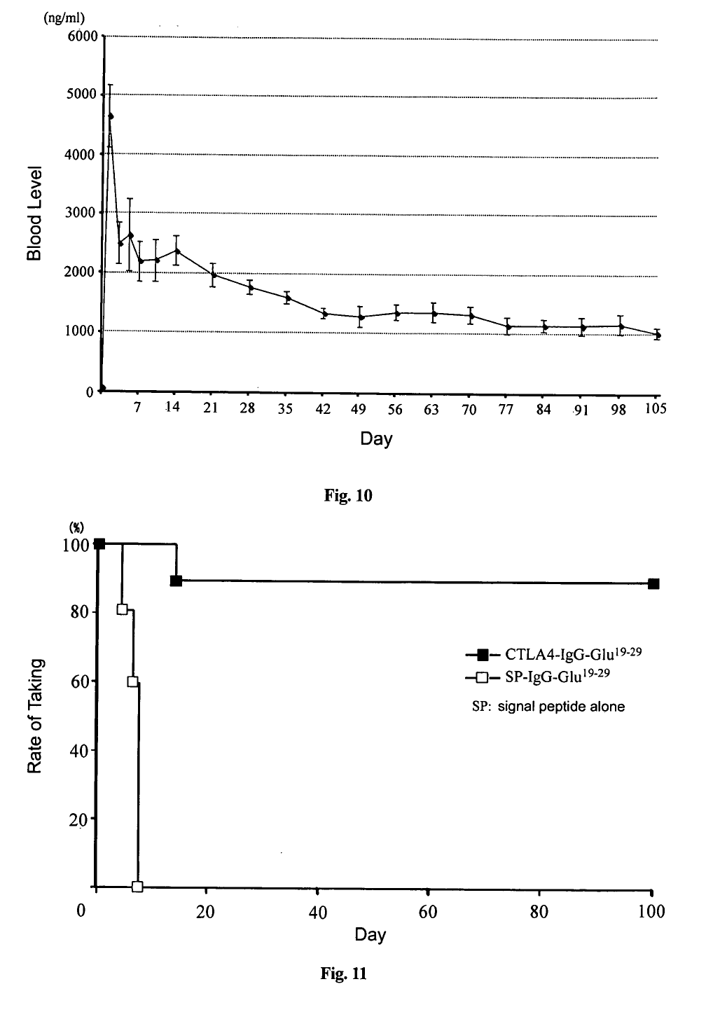

The results are shown in FIGS. 10 and 11. As shown in FIG. 10, in Example 2, the CTLA4-IgG-glucagon¹⁹⁻²⁹ protein level changed as shown in FIG. 7. That is, although the glucagon in a 100-fold diluted sample could not be measured before the administration, the level sharply increased on Day 1 to a protein level of exceeding 5000 ng/ml. Although the protein level gradually decreased thereafter, the protein level was kept at more than 1000 ng/ml until the rats were sacrificed for evaluation on Day 105. As shown in FIG. 11, in Example 2, the heart was rejected in one animal among 10 animals on Day 14, in all of the remaining 9 animals, the heart was taken until Day 105 on which evaluation was performed. In the group treated with pCAGGS-SP-IgG-glucagon¹⁹⁻²⁹ not containing CTLA4, the heart of one animal among 5 animals was rejected on Day 5, the heart of one animal was rejected on Day 6, and the hearts of 3 animals were rejected on Day 7. These results show significant effectiveness of the therapy by pCAGGS-CTLA4-IgG-glucagon¹⁹⁻²⁹.

### EXAMPLE 3, COMPARATIVE EXAMPLE 3

PCR was carried out using as a template the cDNA of cultured rat spleen cells stimulated by lectin, and using as primers 5′-gagaattcatttaaatggcactctgggtgactgcagtc-3′ and 5′-gcagcatcgcggccgcgtggccatagcggaaaagttgctt-3′. The obtained PCR product was incorporated into pCAGGS-IgG-glu 19-29 prepared by the method described in Example 1, using SwaI and NotI.

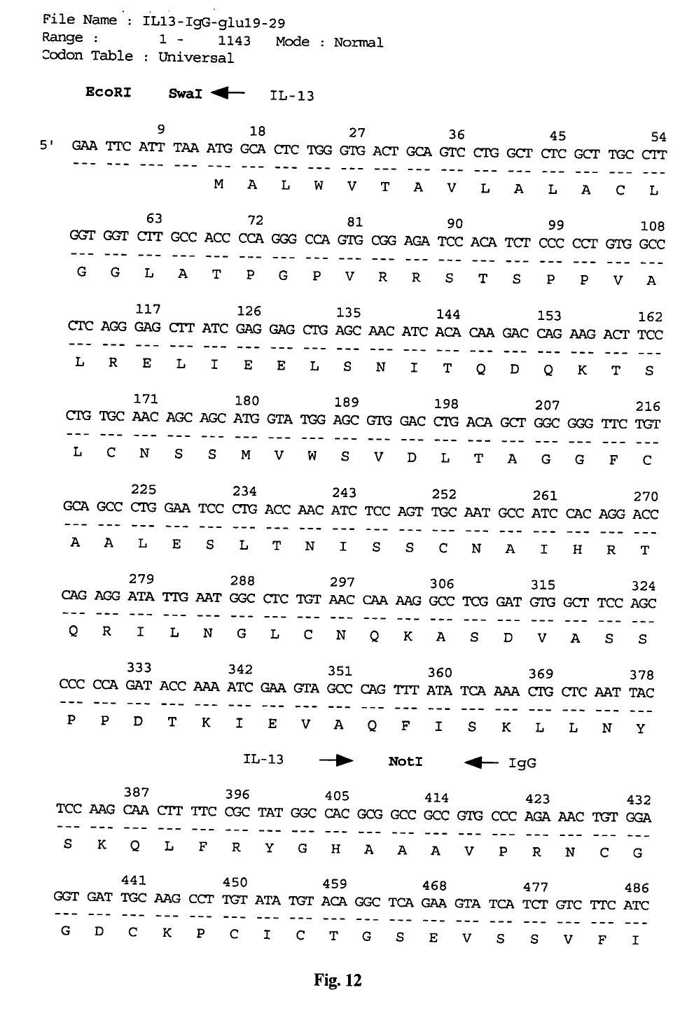

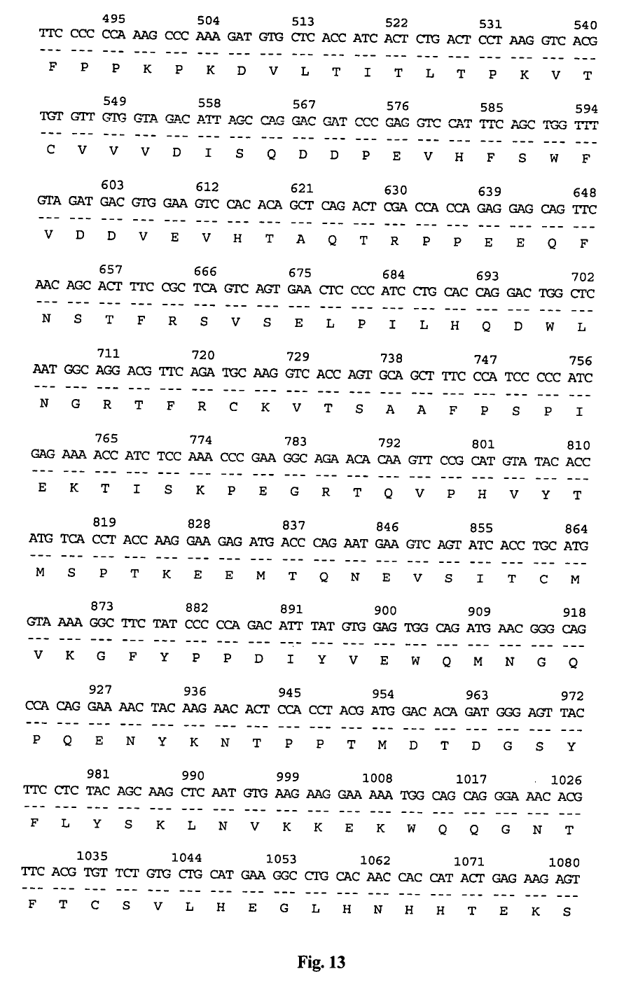

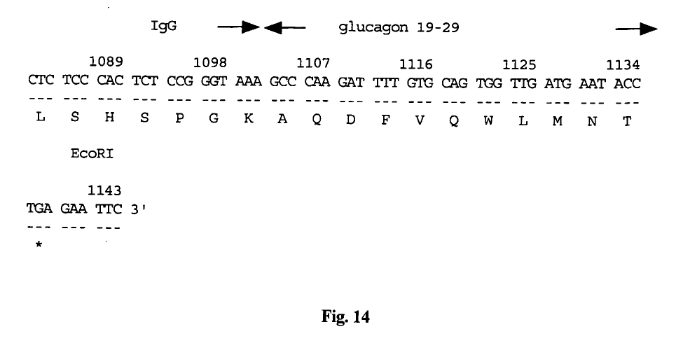

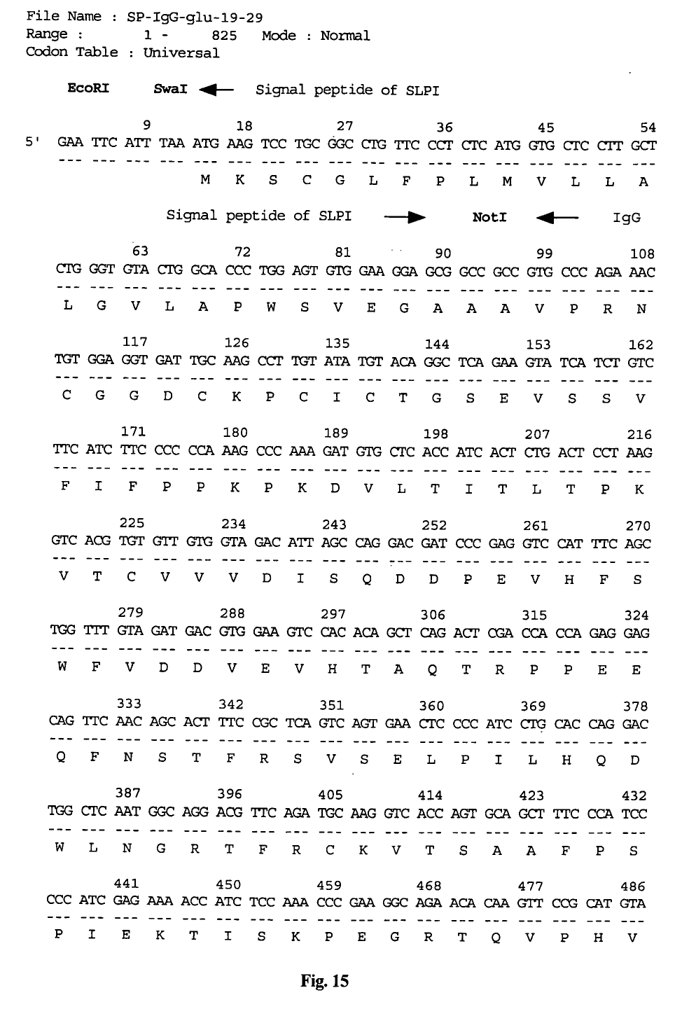

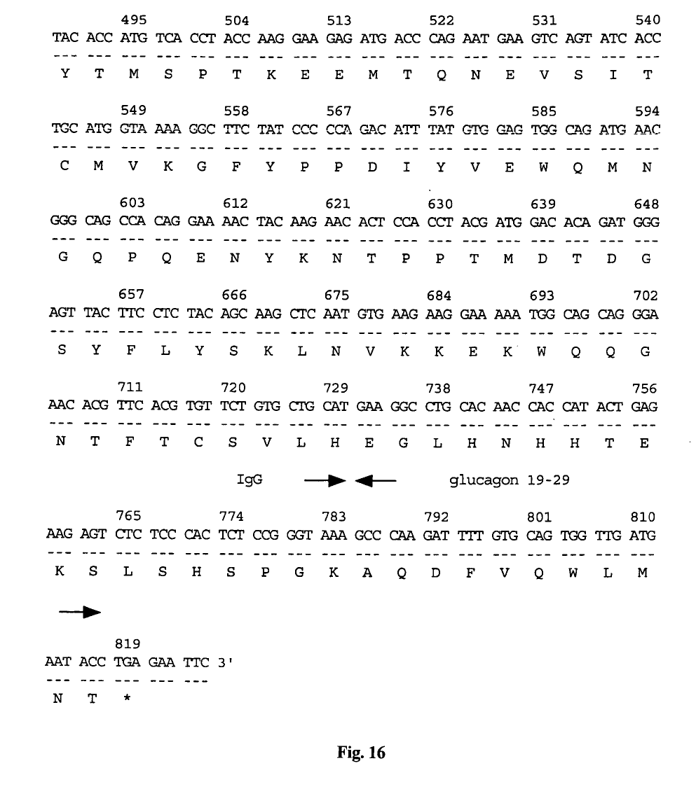

By this, a recombinant vector containing rat IL13-IgG-glucagon¹⁹⁻²⁹ (a nucleic acid fragment in which the rat IgG Fc-coding region was ligated to the downstream of rat interleukin 13 (IL13)-coding region, and the glucagon-originated label peptide-coding region was ligated to the downstream of the rat IgG Fc-coding region) having the nucleotide sequence shown in FIGS. 12 to 14 and in SEQ ID NO:5 (the restriction sites are also shown), inserted into the EcoRI site of the above-described expression vector pCAGGS for mammalian cells, which recombinant vector expresses IL13-IgG-glucagon¹⁹⁻²⁹ in rat cells was prepared (Example 3). For comparison, a recombinant vector in which SP-IgG-glucagon¹⁹⁻²⁹-coding region alone (SEQ ID NO:6 and FIGS. 15 and 16) not containing the IL13-coding region was inserted was also prepared (Comparative Example 3).

To rats suffering from autoimmune myocarditis (A novel experimental model of giant cell myocarditis induced in rats by immunization with cardiac myosin fraction, Clinical Immunology and Immunopathology, November 1990, volume 57, p250-262), the recombinant vector was administered in the same manner as in Example 1, and the blood level was measured. On Day 16 from the administration, the animals were sacrificed and anatomized. The percentage of the area of the myocarditis lesion was also checked.

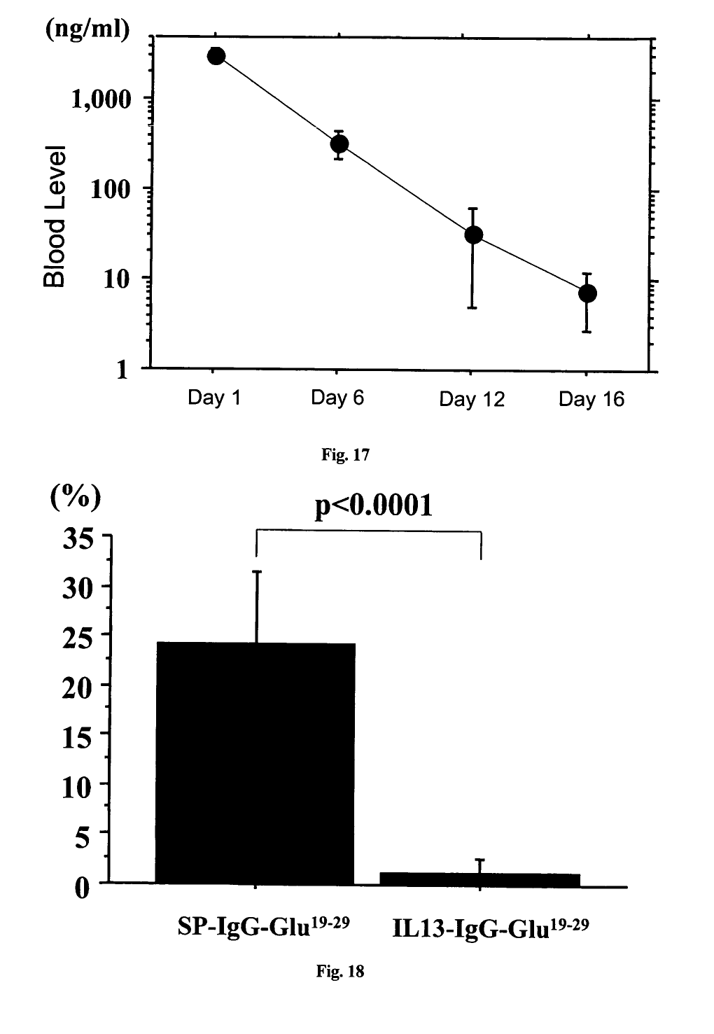

The results are shown in FIGS. 17 and 18. The IL13-IgG-glucagon¹⁹⁻²⁹ protein level changed as shown in FIG. 17. That is, on Day 1, a level exceeding 2000 ng/ml was observed. Although the protein level gradually decreased thereafter, the protein level was kept at more than about 8 ng/ml until the rats were sacrificed for evaluation on Day 16. As shown in FIG. 18, in the group to which pCAGGS-IL13-IgG-glucagon ˜929 (the vector pCAGGS in which IL13-IgG-glucagon¹⁹⁻²⁹ was inserted) that is a vector for gene therapy according to the present invention, the area of the myocarditis lesion was significantly smaller than in the group to which pCAGGS-SP-IgG-glucagon¹⁹⁻²⁹ not containing IL13 was administered. Thus, the effectiveness of the therapy by pCAGGS-IL13-IgG-glucagon¹⁹⁻²⁹ was shown.

### EXAMPLE 4, COMPARATIVE EXAMPLE 4

PCR was carried out using as a template the cDNA of cultured mouse spleen cells stimulated by lectin, and using as primers 5′-gagaattcatttaaatggaaatctgctggggaccctac-3′ and 5′-gcagcatcgcggccgcttggtcftcctggaagtagaactt-3′. The obtained PCR product was incorporated into pCAGGS-IgG-glu19-29 prepared by the method described in Example 1, using SwaI and NotI.

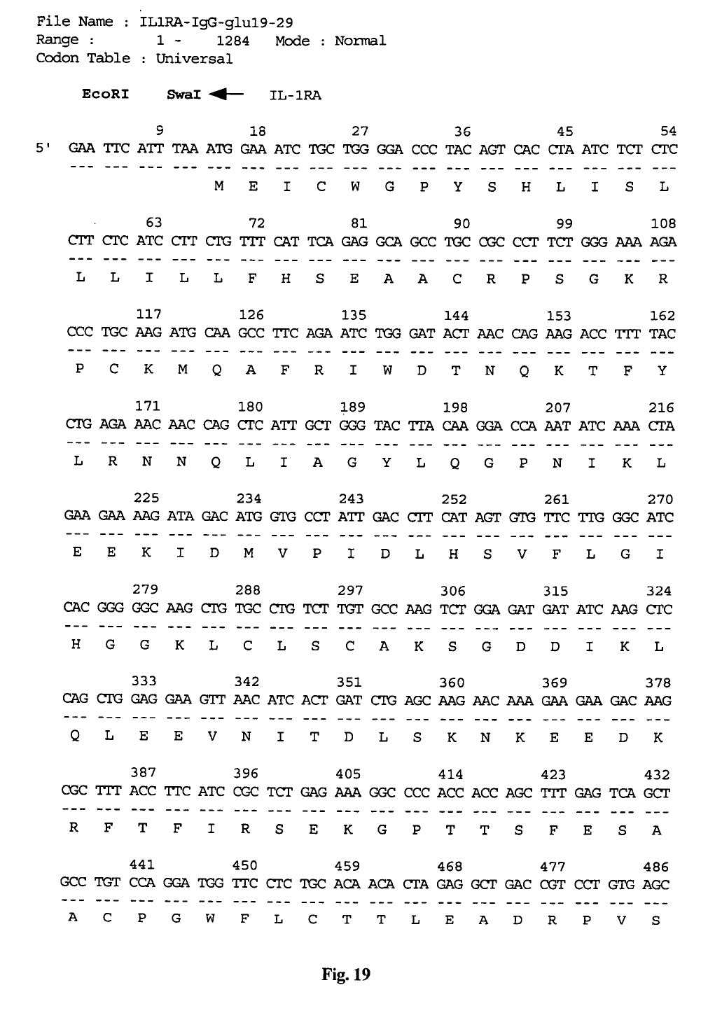

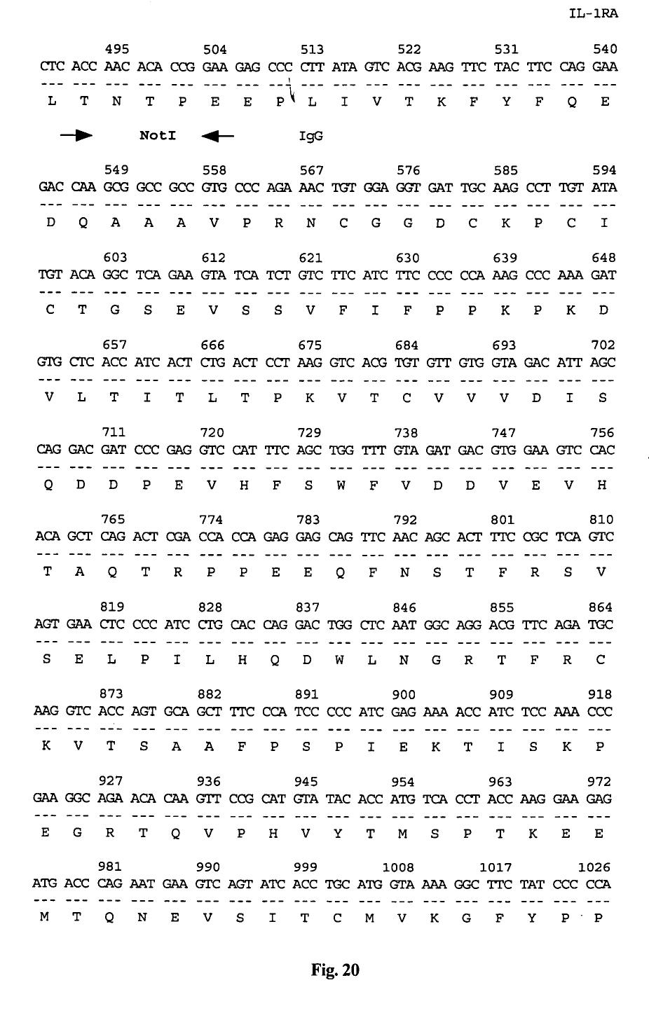

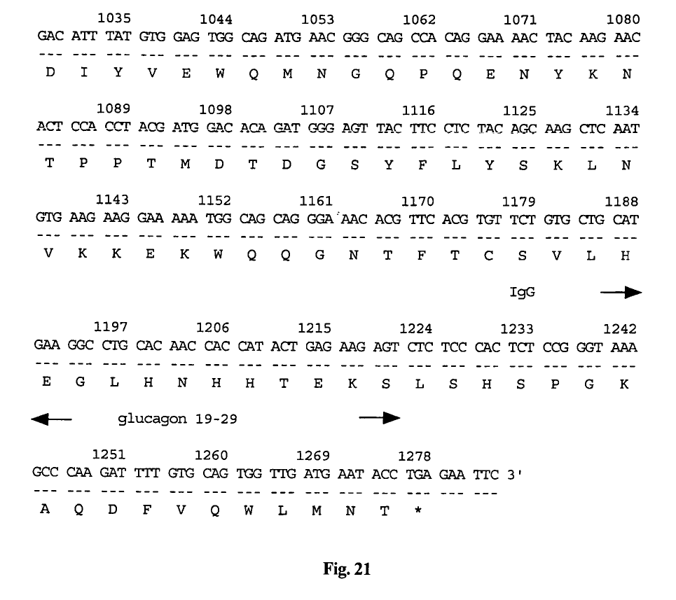

By this, a recombinant vector containing rat IL1RA-IgG-glucagon¹⁹⁻²⁹ (a nucleic acid fragment in which the rat IgG Fc-coding region was ligated to the downstream of interleukin 1 receptor antagonist-coding region, and the glucagon-originated label peptide-coding region was ligated to the downstream of the rat IgG Fc-coding region) having the nucleotide sequence shown in FIGS. 19 to 21 and in SEQ ID NO:7 (the restriction sites are also shown), inserted into the EcoRI site of the above-described expression vector pCAGGS for mammalian cells, which recombinant vector expresses IL1RA-IgG-glucagon¹⁹⁻²⁹ in rat cells was prepared (Example 4).

To rats suffering from autoimmune myocarditis (A novel experimental model of giant cell myocarditis induced in rats by immunization with cardiac myosin fraction, Clinical Immunology and Immunopathology, November 1990, volume 57, p250-262), the recombinant vector was administered in the same manner as in Example 1, and the blood level was measured. On Day 16 from the administration, the animals were sacrificed and anatomized. The percentage of the area of the myocarditis lesion was also checked. For comparison, the above-described vector of Comparative Example 3 was also administered (Comparative Example 4).

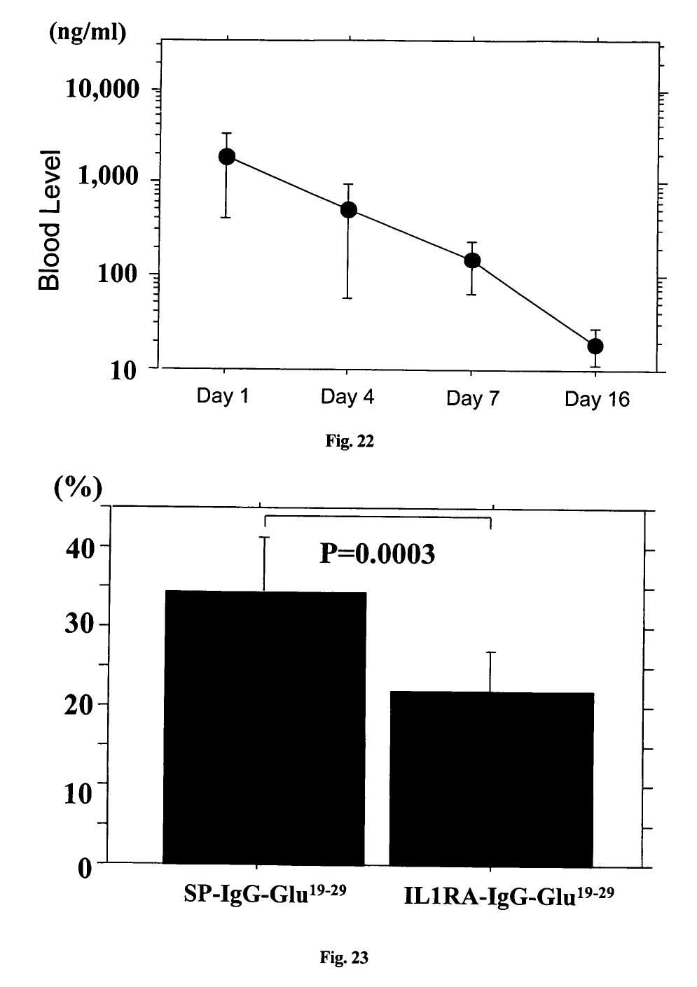

The results are shown in FIGS. 22 and 23. The IL1RA-IgG-glucagon¹⁹⁻²⁹ protein level changed as shown in FIG. 22. That is, on Day 1, a level exceeding 2000 ng/ml was observed. Although the protein level gradually decreased thereafter, the protein level was kept at more than about 20 ng/ml until the rats were sacrificed for evaluation on Day 16. As shown in FIG. 23, in Example 4, the area of the myocarditis lesion was significantly smaller than in Comparative Example 4, so that the effectiveness of the therapy by pCAGGS-IL1RA-IgG-glucagon¹⁹⁻²⁹ was shown.

### EXAMPLE 5

To prepare pCAGGS-glu19-29 having Swal and NotI restriction sites, PCR was performed using as primers 5′-gagaattcatttaaatgagagcggccgccccgggtaaagcccaagattttgtgcagtggttg-3′ and 5′-gagagagagaattctcaggtattcatcaaccactgcacaaaatcttgggc-3′ alone, and the PCR product was inserted into the cloning site of pCAGGS using EcoRI.

Thereafter, PCR was performed using the cDNA of Cos7 cells as a template and using as primers 5′-gagaattcatttaaatgacttccaagctggccgtggct-3′ and 5′-gcagcatcgcggccgctgaattctcagccctcttcaaaaa-3′. The PCR product was incorporated into the pCAGGS-glu19-29 prepared above using SwaI and NotI.

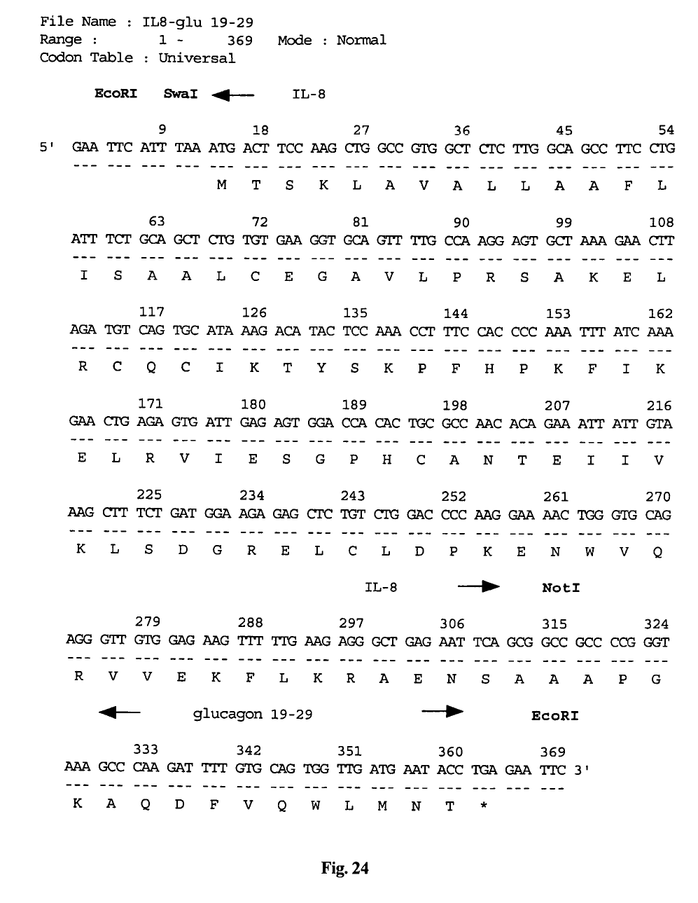

By this, a recombinant vector containing human IL8-glucagon¹⁹⁻²⁹ (a nucleic acid fragment in which the glucagon-originated label peptide-coding region was ligated to the downstream of the human interleukin 8-coding region) having the nucleotide sequence shown in FIG. 24 and in SEQ ID NO:8, inserted into the EcoRI site of the above-described expression vector pCAGGS for mammalian cells, which recombinant vector expresses IL8-glucagon¹⁹⁻²⁹ in rat cells was prepared (Example 5).

The recombinant vector was administered to rats in the same manner as in Example 1. One day after, the blood level was measured. The human IL-8 level in the same sample was also quantified. The quantification of human IL-8 was carried out using IL-8 ELISA kit commercially available from BIOSOURSE (Nivelles, Belgium) in accordance with the Protocols of the product.

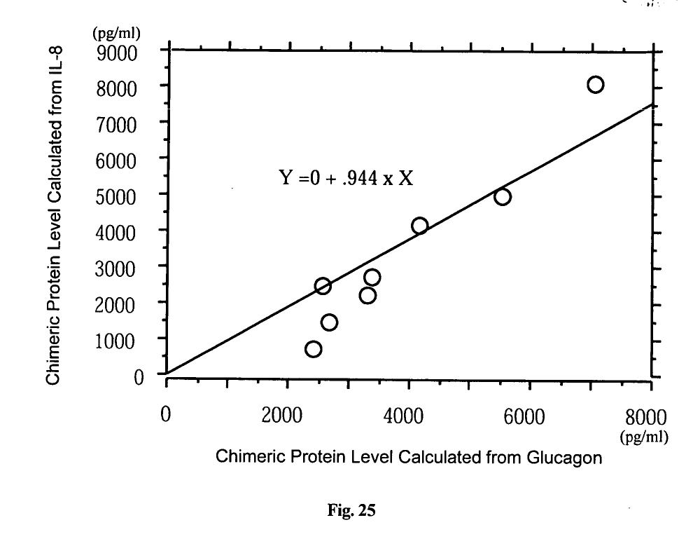

The results are shown in FIG. 25. As shown in FIG. 25, the molar concentrations determined by the both measurements were substantially identical, so that the accuracy of the method using the label peptide of glucagon¹⁹⁻²⁹ was proved.

1. A vector for gene therapy comprising an expression vector for mammalian cells and a nucleic acid coding for a fusion protein of glucagon C-terminal side 19-29 amino acid peptide region and a desired protein region which should be produced in the body, which vector can produce said fusion protein in the mammalian cells.

2. The vector according to claim 1, wherein said glucagon C-terminal side 19-29 amino acid peptide region is ligated to the C-terminal of said desired protein region.

3. The vector according to claim 1 or 2, wherein said desired protein is a cytokine, a fusion protein comprising a cytokine and a constant region of immunoglobulin ligated to said cytokine, a growth factor, a hormone or a cell adhesion factor, or a receptor thereof.

4. The vector according to claim 3, wherein said cytokine or the receptor thereof is selected from the group consisting of interferons and receptors thereof, CTLA4, interleukins and receptors thereof.

5. A method for gene therapy comprising administering an effective amount of said vector for gene therapy according to claim 1 to a mammal or cultured mammalian cells, in which expression of said desired protein in the body or in the cultured mammalian cells is desired.

6. The method according to claim 5, wherein said vector for gene therapy is administered to a mammal.

7. Use of the vector for gene therapy according to claim 1 for the production of a drug for gene therapy.

8. A method for quantifying a desired protein produced in the body or in cultured cells by expression of said vector for gene therapy, comprising quantifying, by immunoassay, said glucagon C-terminal side 19-29 amino acid peptide region in a test sample collected from a mammal or cultured mammalian cells to which said vector for gene therapy according to claim 1 was administered.

9. The method according to claim 8, wherein said test sample is collected from said mammal to which said vector for gene therapy was administered.

10. The method according to claim 9, wherein said test sample is a blood sample.

11. A label for labeling a desired protein produced by expression of an externally administered expression vector in the body of a mammal or in cultured mammalian cells, consisting essentially of glucagon C-terminal side 19-29 amino acid peptide.

12. A label for labeling a desired protein produced by expression of an externally administered expression vector in the body of a mammal, consisting essentially of glucagon C-terminal side 19-29 amino acid peptide.

13. A method for labeling a protein produced in the body or in cultured cells, comprising labeling a desired protein produced in the body or in cultured cells with glucagon C-terminal side 19-29 amino acid peptide by expressing said desired protein produced by expression of an externally administered expression vector in the body of a mammal or in cultured mammalian cells, as a fusion protein with said glucagon C-terminal side 19-29 amino acid peptide as a label.

14. The method according to claim 13, comprising labeling said desired protein produced in the body with glucagon C-terminal side 19-29 amino acid peptide by expressing said desired protein produced by expression of an externally administered expression vector in the body of a mammal, as a fusion protein with said glucagon C-terminal side 19-29 amino acid peptide as a label.

15. Use of glucagon C-terminal side 19-29 amino acid peptide as a label for a desired protein produced by expression of an externally administered expression vector in the body of a mammal or in cultured mammalian cells.

16. Use of glucagon C-terminal side 19-29 amino acid peptide as a label for a desired protein produced by expression of an externally administered expression vector in the body of a mammal.
